(19) United States
(12) Patent Application Publication
(10) Pub. No.: US 2014/0179633 A1
(43) Pub. Date: Jun. 26, 2014

(54) COMPOSITION COMPRISING PYRAZINO-TRIAZINE DERIVATIVES

(71) Applicants: Min-Seok Choi, Gyeonggi-do (KR); Young-Hoon Kim, Seoul (KR)

(72) Inventors: Min-Seok Choi, Gyeonggi-do (KR); Young-Hoon Kim, Seoul (KR)

(73) Assignee: JW PHARMACEUTICAL CORPORATION, Seoul (KR)

(21) Appl. No.: 14/238,644

(22) PCT Filed: Aug. 24, 2012
(86) PCT No.: PCT/KR2012/006774

§ 371 Date: Feb. 12, 2014

Related U.S. Application Data
(60) Provisional application No. 61/555,427, filed on Nov. 3, 2011.

(30) Foreign Application Priority Data
Aug. 26, 2011 (KR) 10-2011-0085493

(51) Int. Cl. A61K 31/675; A61K 47/12; A61K 47/26; A61K 47/10; A61K 47/22; A61K 47/34; A61K 47/02; A61K 47/18

(52) U.S. Cl. CPC A61K 31/675; A61K 47/02; A61K 47/12; A61K 47/183; A61K 47/10; A61K 47/22; A61K 47/34; A61K 47/26
USPC 514/81

(57) ABSTRACT

Disclosed is a composition including a pyrazino-triazine derivative and a pharmaceutically acceptable salt thereof. The composition includes a solubilizer or a stabilizer and thus can exhibit very excellent solubility and stability.

### BACKGROUND OF THE INVENTION

1. Technical Field

The present invention relates to a composition comprising a pyrazino-triazine derivative as an active ingredient, which is easily solubilized and has very excellent stability.

2. Description of the Related Art

The random screening of molecules for their possible activity as therapeutic agents has been carried out for many years, and a number of important drugs have been consequently discovered.

Among these, WO10/120,112, U.S. Pat. No. 7,576,084, U.S. Pat. No. 7,566,711, U.S. Pat. No. 7,671,054, and WO09/148,192 disclose a variety of compounds having a pyrazino-triazine derivative form, which may be used as anticancer agents.

Particularly, it is disclosed in WO10/120,112 that the compound includes a prodrug functional group in the structure thereof and thus is improved in terms of solubility and stability. However, in the case of injections formulated in the manner described in this patent, a pyrazino-triazine derivative does not dissolve well enough, or there is a probability of the compound degrading over time in an aqueous state. Such problems may incur the generation and precipitation of degraded products, undesirably causing vessel pain, vessel obstruction or phlebitis due to the precipitation upon their administration into a vein. Further, in the case of medical compositions, it is important to minimize the generation of related substances with the passage of time. According to the ICH guidelines, regulations state that when the amount of the generated related substance is 0.1% or more in the case where the maximum dosage of the medical composition per day is 1 g or less, the corresponding related substance must be reported. Therefore, it is actually required to prepare a composition in which a pyrazino-triazine derivative is easily dissolved such that it is sufficiently used as an injection and is stable even when it is stored for a long period of time, and in which the content of related substances is minimized.

Accordingly, the present inventors discovered the fact that when a stabilizer and/or a solubilizer is added to the pyrazino-triazine derivative, the solubility and stability of the pyrazino-triazine derivative may be remarkably increased, thus facilitating the formation of a formulation, and in particular enabling the supply of an aqueous solution that is stable upon storage at room temperature, thereby culminating in the present invention. Furthermore, room-temperature stability is very favorable in terms of storage and transport.

### SUMMARY OF THE INVENTION

Accordingly, the present invention has been made to solve the above-mentioned problems, and an object of the present invention is to provide a composition which can easily be made into a formulation by improving the solubility and stability of a pyrazino-triazine derivative.

An aspect of the present invention provides a composition, comprising: at least one compound selected from the group consisting of a compound represented by Chemical Formula 1 below, an isomer thereof and a pharmaceutically acceptable salt thereof, as an active ingredient, wherein the composition comprises a solubilizer and a stabilizer, wherein the solubilizer is one or more selected from the group consisting of a saccharide, an alcohol, an acid, a salt and a polymer and the stabilizer including one or more selected from the group consisting of a saccharide, an acid, a salt, an antioxidant, and a polymer:

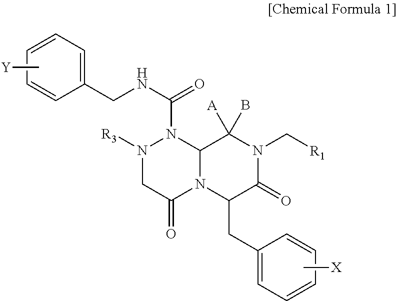

wherein R₁ is substituted or unsubstituted C3˜C10 aryl or substituted or unsubstituted C3˜C10 heteroaryl with at least one nitrogen (N) atom; R₃ is hydrogen, C1˜C6 alkyl, C2˜C6 alkenyl, C2˜C6 alkynyl, C3˜C10 heteroaryl with at least one nitrogen (N) atom, C3˜C10 arylalkyl or C3˜C10 heteroarylalkyl with at least one nitrogen (N) atom; A is hydrogen or C1˜C6 alkyl; B is hydrogen or C1˜C6 alkyl; X is —O—PO₃H₂, —O—SO₂NH₂, carbamate,

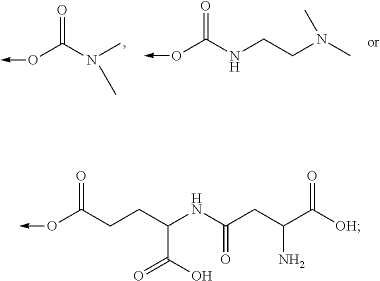

and Y is hydrogen, C3˜C10 aryl, C3˜C10 heteroaryl with at least one nitrogen (N) atom or C1˜C6 alkyl. In an embodiment of the present invention, the substituted C3˜C10 aryl or the substituted C3˜C10 heteroaryl with at least one nitrogen (N) atom may be substituted with C1˜C6 alkyl or

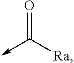

wherein Ra is hydrogen or an C1˜C6 alkyl.

In another embodiment of the present invention, R₁ may be naphthyl, quinolinyl, indolyl, indazolyl, substituted naphthyl, substituted quinolinyl, substituted indolyl or substituted indazolyl, wherein the substituted naphthyl, the substituted the quinolinyl, the substituted indolyl and the substituted indazolyl have at least one substituent selected from C1˜C6 alkyl or

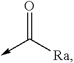

wherein Ra is hydrogen or C1˜C6alkyl; R₃ may be methyl or propenyl; either or both of A and B may be hydrogen; X may be —O—PO₃H₂; and Y may be hydrogen.

In still another embodiment of the present invention, R₁ may be

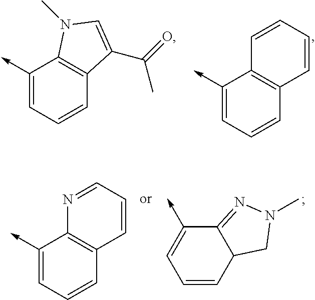

### R₃ may be

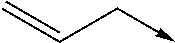

or a methyl group; either or both of A and B may be hydrogen; X may be —O—PO₃H₂; and Y is hydrogen.

In still another embodiment of the present invention, the active ingredient may be a compound represented by one of Chemical Formulas 1-1 to 1-3 below:

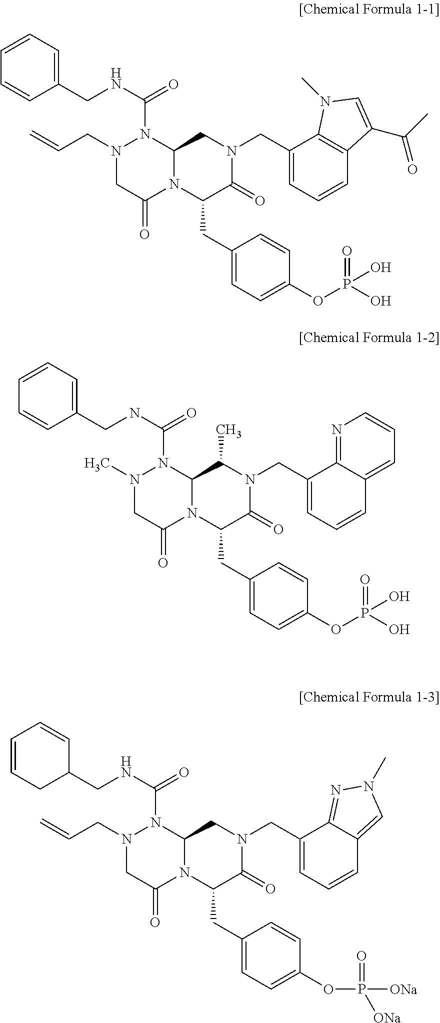

In still another embodiment of the present invention, the active ingredient may be at least one compound selected from the group consisting of a compound represented by one of Chemical Formulas 2 to 6 below, an isomer thereof and a pharmaceutically acceptable salt thereof:

| No. | Chemical Formula |
|---|---|
| 2 | 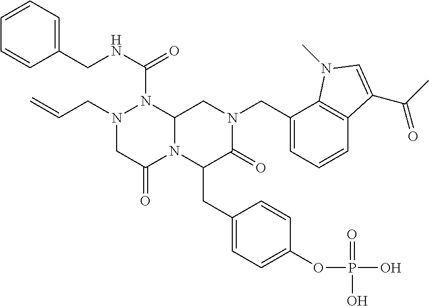 [SMILES: C=CCN1CC(=O)N2C(Cc3ccc(OP(=O)(O)O)cc3)C(=O)N(Cc3cccc4c(C(C)=O)cn(C)c34)CC2N1C(=O)NCc1ccccc1] |
| 3 | 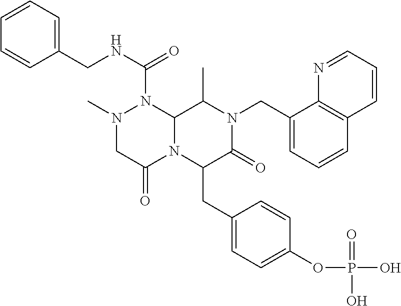 [SMILES: CC1C2N(C(=O)CN(C)N2C(=O)NCc2ccccc2)C(Cc2ccc(OP(=O)(O)O)cc2)C(=O)N1Cc1cccc2cccnc12] |
| 4 | 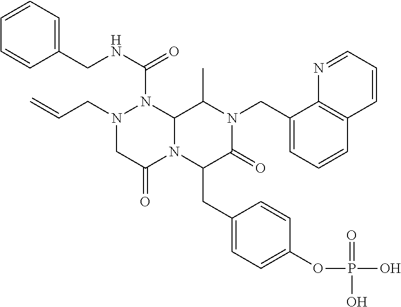 [SMILES: C=CCN1CC(=O)N2C(Cc3ccc(OP(=O)(O)O)cc3)C(=O)N(Cc3cccc4cccnc34)C(C)C2N1C(=O)NCc1ccccc1] |
| 5 | 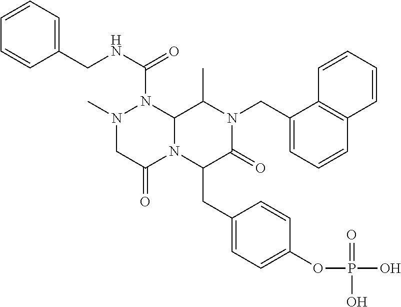 [SMILES: CC1C2N(C(=O)CN(C)N2C(=O)NCc2ccccc2)C(Cc2ccc(OP(=O)(O)O)cc2)C(=O)N1Cc1cccc2ccccc12] |
| 6 | 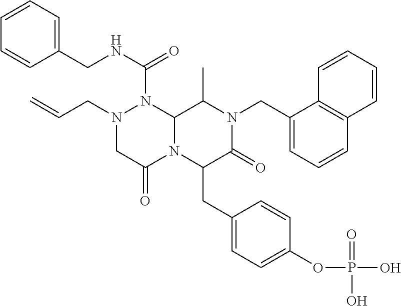 [SMILES: C=CCN1CC(=O)N2C(Cc3ccc(OP(=O)(O)O)cc3)C(=O)N(Cc3cccc4ccccc34)C(C)C2N1C(=O)NCc1ccccc1] |

In still another embodiment of the present invention, the stabilizer may comprises at least one selected from saccharide of mannitol, sucrose, lactose, glucose, hydroxypropyl beta cyclodextrin (HP-B-CD) or sorbitol; acid of one or more organic acids selected from benzene sulfonic acid, benzoic acid, citric acid, lactic acid, maleic acid, methane sulfonic acid, succinic acid, tartaric acid and gentisic acid, one or more inorganic acids selected from hydrochloric acid, phosphoric acid, hydrogen bromide and sulfuric acid, one or more amino acids selected from glutamine, asparagine, leucine, glycine, isoleucine, threonine, phenylalanine, histidine, cysteine and lycine, or fatty acids; salt of sodium chloride, sodium citrate, sodium sulfite, calcium chloride, disodium edentate, dibasic sodium phosphate, dibasic sodium phosphate dihydrate, monobasic sodium phosphate dihydrate, sodium bicarbonate, disodium succinate, gentisic acid ethanolamine, ammonium hydroxide, sodium benzoate, sodium dithionite, sodium glutamate, sodium lactate, sodium metabisulfite, sodium tartrate, sodium thioglycolate or zinc chloride; antioxidant of acetone sodium bisulfate, sodium bisulfate, butylated hydroxyl anisole (BHA), butylated hydroxyl toluene (BHT), sodium formaldehyde sulfoxylate, monothioglycerol (thioglycerol), propyl gallate, vitamin C, ethylenediamine tetraacetic acid (EDTA) or tocopherol; and polymer of polyethylene glycol, polysorbate, polyoxypropylene, a polyethylene-propylene glycol copolymer or polyoxyethylene sorbitan monooleate.

In still another embodiment of the present invention, the solubilizer may comprises at least one selected from: saccharide of mannitol, sucrose, lactose, sorbitol, microcrystalline cellulose, or hydroxypropyl beta-cyclodextrin (HP-B-CD); alcohol of benzyl alcohol, glycerol, isopropanol, propylene glycol, or ethanol; acid of one or more organic acids selected from citric acid and lactic acid, hydroxychloric acid, one or more amino acids selected from L-arginine and L-glycine, or stearic acid; salt of sodium bicarbonate, sodium carbonate, sodium citrate, sodium hydroxide, sodium acetate, sodium chloride, sodium borate, sodium sulfite, calcium carbonate, potassium citrate, sodium desoxycholate, or disodium edatate; and polymer of polyethylene glycol, polysorbate, polyoxypropylene, polyoxyethylene, polyoxyethylated fatty acid, polyvinylpyrrolidone, polyoxyl castor oil, a polyethylene-propylene glycol copolymer, polyoxyethyleneglyceroltriricinolate, polyethoxylated castor oil, or polyoxyethylene sorbitan monooleate.

In still another embodiment of the present invention, the stabilizer may be at least one selected from the group consisting of a saccharide, a sodium salt and a polyethylene-propylene glycol copolymer.

In still another embodiment of the present invention, the saccharide may be at least one selected from the group consisting of mannitol, sucrose and sorbitol, and the sodium salt may be at least one selected from the group consisting of sodium chloride, sodium citrate and sodium sulfite.

In still another embodiment of the present invention, the stabilizer may be at least one selected from the group consisting of sodium chloride, sucrose, mannitol and sorbitol.

In still another embodiment of the present invention, the stabilizer may be sucrose.

In still another embodiment of the present invention, the weight ratio of the active ingredient to the stabilizer may be 1:0.02˜30.

In still another embodiment of the present invention, the composition may be a liquid injection dosage form.

Another aspect of the present invention provides a method of stabilizing a composition comprising at least one compound selected from the group consisting of a compound represented by Chemical Formula 1 below, an isomer thereof and a pharmaceutically acceptable salt thereof, as an active ingredient, comprising the step of mixing the composition with at least one stabilizer selected from the group consisting of a saccharide, an acid, a salt, an antioxidant and a polymer:

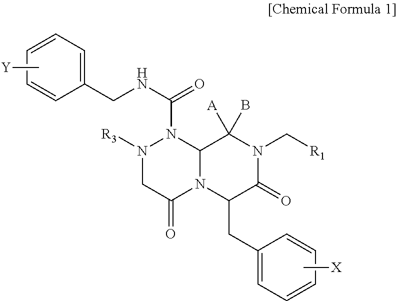

wherein R₁ is substituted or unsubstituted C3˜C10 arylor substituted or unsubstituted C3˜C10 heteroaryl with at least one nitrogen (N) atom; R₃ is hydrogen, C1˜C6 alkyl, C2˜C6 alkenyl, C2˜C6 alkynyl, C3˜C10 heteroaryl with at least one nitrogen (N) atom, C3˜C10 arylalkyl or C3˜C10 heteroarylalkyl with at least one nitrogen (N) atom; A is hydrogen or C1˜C6 alkyl; B is hydrogen or C1˜C6 an alkyl; X is —O—PO₃H₂, —O—SO₂NH₂, carbamate,

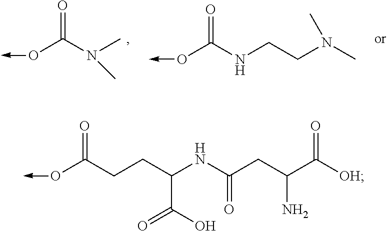

and Y is hydrogen, C3˜C10 aryl, C3˜C10 heteroaryl with at least one nitrogen (N) atom or C1˜C6 alkyl.

### Advantage of the Invention

As described hitherto, the composition according to the present invention can be remarkably improved in terms of solubility and stability and can thus be very easily formulated. In particular, the development of a liquid injection dosage form is possible, thus preventing the problems of powder injections or lyophilization injections, for example, medical incidents occurring upon pretreatment for filtration or drug dissolution before administration. Furthermore, stability can be maintained at room temperature thus facilitating storage and transport.

### DESCRIPTION OF THE PREFERRED EMBODIMENTS

The present invention pertains to a composition which includes a pyrazino-triazine derivative as an active ingredient, a solubilizer and a stabilizer and thus which exhibits improved solubility and stability and is easily formulated.

The present inventors found and proved that when a solubilizer and a stabilizer is added to the pyrazino-triazine derivative of the present invention, solubility and stability may be remarkably increased, making it very easy to form a formulation, which culminated in the present invention.

As used herein, the term “pyrazino-triazine derivative” refers to a compound derivative represented by Chemical Formula 1 below.

In detail, the pyrazino-triazine derivative according to the present invention may be at least one compound selected from the group consisting of a compound represented by Chemical Formula 1 below, an isomer thereof and a pharmaceutically acceptable salt thereof, but is not limited thereto.

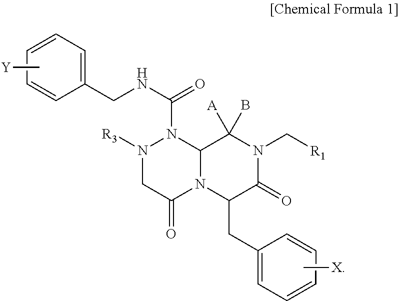

In Chemical Formula 1, R₁ may be substituted or unsubstituted C3˜C10 arylor substituted or unsubstituted C3˜C10 heteroaryl with at least one nitrogen (N) atom.

Here, the substituted C3˜C10 aryl or the substituted C3˜C10 heteroaryl with at least one nitrogen (N) atom may be substituted with C1˜C6 alkyl or

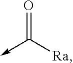

wherein Ra is hydrogen or C1˜C6 alkyl.

Preferably, R₁ may be

naphthyl, quinolinyl, indolyl, indazolyl, substituted naphthyl, substituted quinolinyl, substituted indolyl or substituted indazolyl, wherein the substituted naphthyl, the substituted the quinolinyl, the substituted indolyl and the substituted indazolyl have at least one substituent selected from C1˜C6 alkyl or

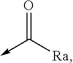

wherein Ra is hydrogen or C1˜C6 alkyl. More preferably, R₁ may be any one of

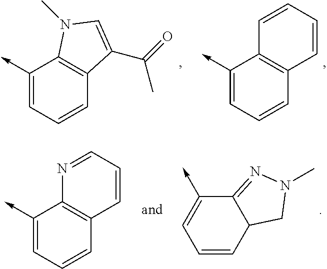

In Chemical Formula 1, R₃ may be hydrogen, C1˜C6 alkyl, C2˜C6 alkenyl, C2˜C6 alkynyl, C3˜C10 heteroaryl with at least one nitrogen (N) atom, C3˜C10 arylalkyl or C3˜C10 heteroarylalkyl with at least one nitrogen (N) atom. Preferably, R₃ may be a methyl or a propenyl

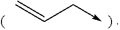

In Chemical Formula 1, A may be hydrogen or C1˜C6 alkyl, B may be hydrogen or C1˜C6 alkyl, and either or both of A and B may be hydrogen.

In Chemical Formula 1, X may be —O—PO₃H₂, —O—SO₂NH₂, carbamate,

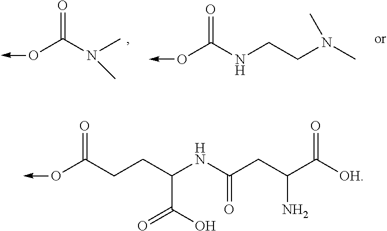

### Preferably, X may be —O—PO₃H₂.

In Chemical Formula 1, Y may be hydrogen, C3˜C10 aryl, C3˜C10 heteroaryl with at least one nitrogen (N) atom or C1˜C6 alkyl. Preferably, Y may be hydrogen.

Further, the pyrazino-triazine derivative of the present invention may be at least one compound selected from the group consisting of a compound represented by one of Chemical Formulas 1-1 to 1-3 below, an isomer thereof and a pharmaceutically acceptable salt thereof:

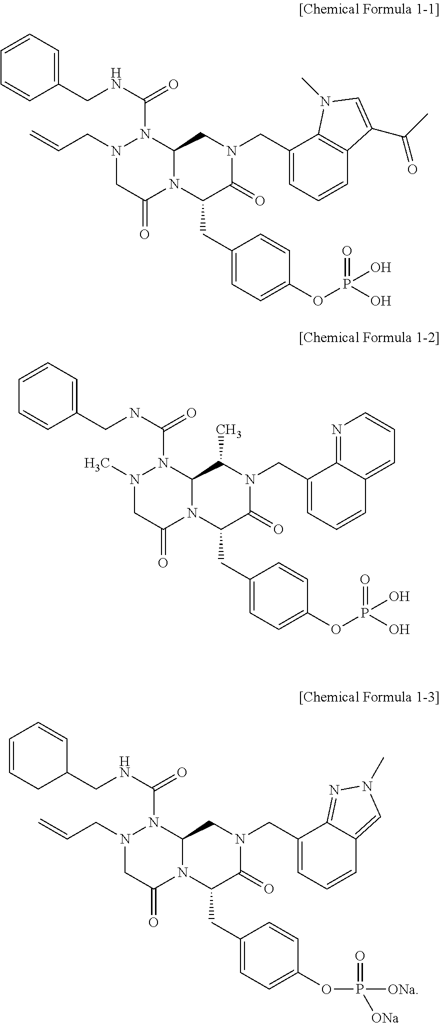

Further, the pyrazino-triazine derivative of the present invention may be at least one compound selected from the group consisting of a compound represented by one of Chemical Formulas 2 to 6 below, an isomer thereof and a pharmaceutically acceptable salt thereof, but is not limited thereto:

| No. | Chemical Formula |
|---|---|
| 2 | 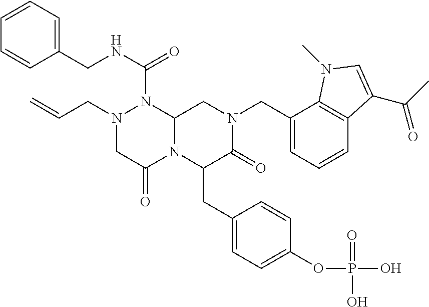 [SMILES: C=CCN1CC(=O)N2C(Cc3ccc(OP(=O)(O)O)cc3)C(=O)N(Cc3cccc4c(C(C)=O)cn(C)c34)CC2N1C(=O)NCc1ccccc1] |
| 3 | 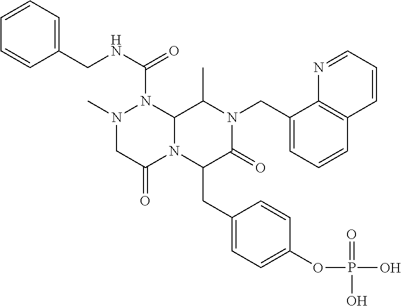 [SMILES: CC1C2N(C(=O)CN(C)N2C(=O)NCc2ccccc2)C(Cc2ccc(OP(=O)(O)O)cc2)C(=O)N1Cc1cccc2cccnc12] |
| 4 | 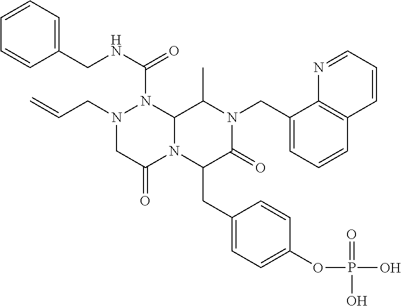 [SMILES: C=CCN1CC(=O)N2C(Cc3ccc(OP(=O)(O)O)cc3)C(=O)N(Cc3cccc4cccnc34)C(C)C2N1C(=O)NCc1ccccc1] |
| 5 | 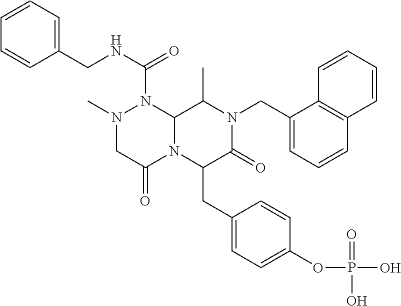 [SMILES: CC1C2N(C(=O)CN(C)N2C(=O)NCc2ccccc2)C(Cc2ccc(OP(=O)(O)O)cc2)C(=O)N1Cc1cccc2ccccc12] |
| 6 | 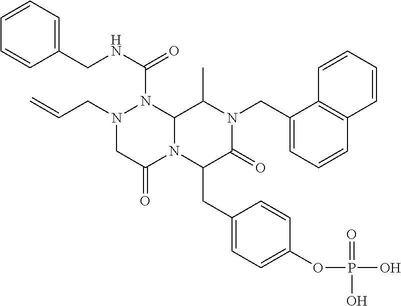 [SMILES: C=CCN1CC(=O)N2C(Cc3ccc(OP(=O)(O)O)cc3)C(=O)N(Cc3cccc4ccccc34)C(C)C2N1C(=O)NCc1ccccc1] |

The pyrazino-triazine derivative of the present invention may be prepared using the method disclosed in WO12/050,393, WO10/120,112, WO09/051,397 or WO09/148,192. Further, the pyrazino-triazine derivative of the present invention may be prepared according to Scheme 1 below.

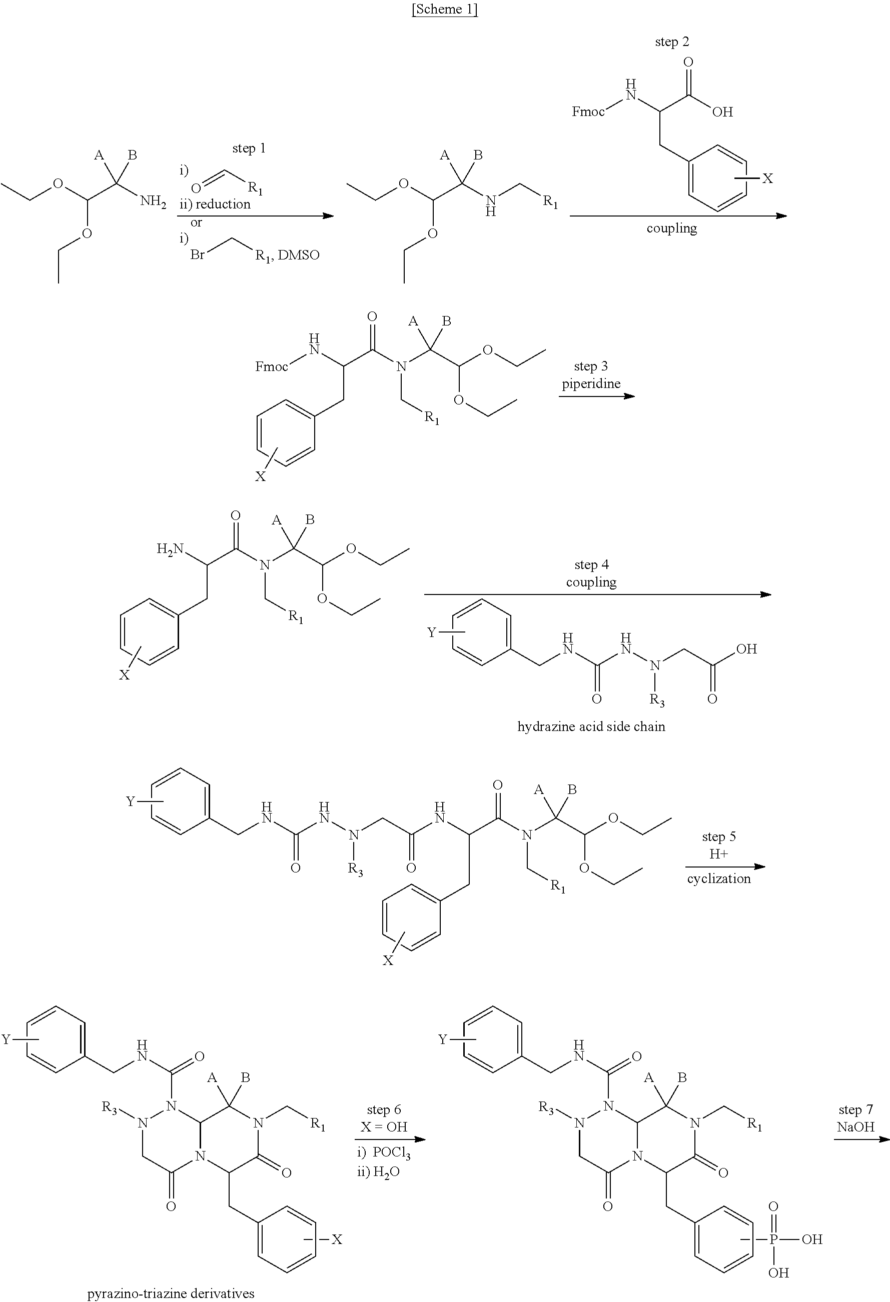

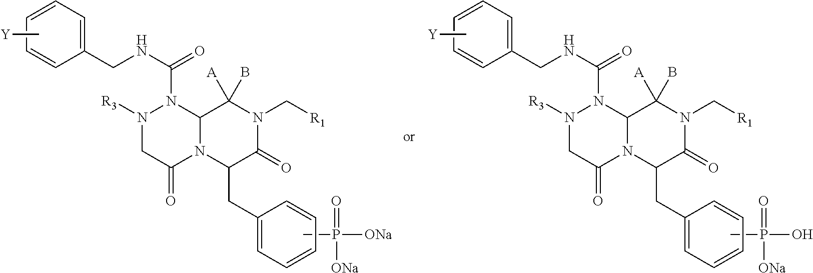

In Scheme 1, A, B, R₁, R₃, X and Y are each as defined above.

The reaction of Scheme 1 is briefly described below.

Step 1: reacting substituted aminoacetal with aldehyde or alkyl halogen;

Step 2: coupling the resulting substituted aminoacetal of step 1 with an amino acid to form a peptide;

Step 3: deprotecting the peptide of step 2 with a salt;

Step 4: coupling the deprotected peptide of step 3 with a hydrazine acid side chain;

Step 5: cyclizing the resulting peptide of step 4 under an acidic condition to form a pyrazino-triazine derivative;

Step 6: introducing an X substituent into the pyrazino-triazine derivative of step 5; and

Step 7: introducing a salt into the X substituent of step 6.

In the present invention, the term “pharmaceutically acceptable salt” refers to a salt used typically in the pharmaceutical field. Examples of the pharmaceutically acceptable salt include sodium salts, magnesium salts, calcium salts and potassium salts, but are not limited thereto. Particularly useful is a sodium salt.

The composition of the present invention includes a stabilizer and a solubilizer.

The stabilizer of the present invention may be a saccharide, an acid, a salt, an antioxidant or a polymer, but is not limited thereto.

Specifically, the stabilizer may be at least one saccharide selected from the group consisting of mannitol, sucrose, lactose, glucose, hydroxypropyl beta cyclodextrin (HP-B-CD) and sorbitol; at least one acid selected from the group consisting of one or more organic acids selected from benzene sulfonic acid, benzoic acid, citric acid, lactic acid, maleic acid, methane sulfonic acid, succinic acid, tartaric acid and gentisic acid, one or more inorganic acids selected from hydrochloric acid, phosphoric acid, hydrogen bromide and sulfuric acid, one or more amino acids selected from glutamine, asparagine, leucine, glycine, isoleucine, threonine, phenylalanine, histidine, cysteine and lycine, and fatty acids; at least one salt selected from the group consisting of sodium chloride, sodium citrate, sodium sulfite, calcium chloride, disodium edentate, dibasic sodium phosphate, dibasic sodium phosphate dihydrate, monobasic sodium phosphate dihydrate, sodium bicarbonate, disodium succinate, gentisic acid ethanolamine, ammonium hydroxide, sodium benzoate, sodium dithionite, sodium glutamate, sodium lactate, sodium metabisulfite, sodium tartrate, sodium thioglycolate and zinc chloride; at least one antioxidant selected from the group consisting of acetone sodium bisulfate, sodium bisulfate, butylated hydroxyl anisole (BHA), butylated hydroxyl toluene (BHT), sodium formaldehyde sulfoxylate, monothioglycerol (thioglycerol), propyl gallate, vitamin C, ethylenediamine tetraacetic acid (EDTA) and tocopherol; or at least one polymer selected from the group consisting of polyethylene glycol, polysorbate, polyoxypropylene, a polyethylene-propylene glycol copolymer and polyoxyethylene sorbitan monooleate.

Preferably, the stabilizer may be at least one saccharide selected from the group consisting of mannitol, sucrose and sorbitol; at least one acid selected from the group consisting of organic acids, amino acids such as glutamine, asparagine and the like, and fatty acids; at least one sodium salt selected from the group consisting of sodium chloride, sodium citrate and sodium sulfite; at least one antioxidant selected from the group consisting of vitamin C and tocopherol; or a polyethylene-propylene glycol copolymer.

More preferably, the stabilizer may be at least one selected from the group consisting of a saccharide, a sodium salt and a polyethylene-propylene glycol copolymer. In this case, the saccharide may be at least one selected from the group consisting of mannitol, sucrose and sorbitol, and the sodium salt may be at least one selected from the group consisting of sodium chloride, sodium citrate and sodium sulfite.

Most preferably, the stabilizer may be at least one selected from the group consisting of sodium chloride, sucrose, mannitol and sorbitol.

The weight ratio of the stabilizer to the active ingredient may be in the range of 1:0.02˜30, but is not limited thereto. The case where the stabilizer is added in an amount more than the above upper limit may undesirably cause problems attributable to increased viscosity and toxicity of the stabilizer itself In contrast, in the case where the stabilizer is added in an amount less than the above lower limit, stability is not ensured under storage conditions, and drugs that have had their effect diminished while being stored may be administered into a patient. As well, the toxicity attributable to the degraded products or the toxicity attributable to precipitation or deterioration may occur, and there may be problems caused with the blood vessels depending on the properties of the injections.

The amount of the added stabilizer may be determined by referring to a general technical level in the range in which the stability of the compound can be exhibited, and may also be determined with reference to the maximum daily amount of each component that can be used as a stabilizer. Preferably, the weight ratio of sodium chloride to the active ingredient may be 1:0.5 or more, the weight ratio of sucrose to the active ingredient may be 1:0.4 or more, the weight ratio of sorbitol to the active ingredient may be 1:0.5 or more, the weight ratio of mannitol to the active ingredient may be 1:1 or more, the weight ratio of sodium citrate to the active ingredient may be 1:0.04 or more, the weight ratio of sodium sulfite to the active ingredient may be 1:0.02 or more, and the weight ratio of a polyethylene-propylene glycol copolymer to the active ingredient may be 1:0.03 or more.

The solubilizer of the present invention may be a saccharide, an alcohol, an acid, a salt or a polymer, but is not limited thereto.

Specifically, the solubilizer may be at least one saccharide selected from the group consisting of at least one saccharide selected from the group consisting of mannitol, sucrose, lactose, sorbitol, microcrystalline cellulose and hydroxypropyl beta-cyclodextrin (HP-B-CD); at least one alcohol selected from the group consisting of benzyl alcohol, glycerol, isopropanol, propylene glycol, and ethanol; at least one acid selected from the group consisting of one or more organic acids selected from citric acid and lactic acid, hydroxychloric acid, one or more amino acids selected from L-arginine and L-glycine, and stearic acid; at least one salt selected from the group consisting of sodium bicarbonate, sodium carbonate, sodium citrate, sodium hydroxide, sodium acetate, sodium chloride, sodium borate, sodium sulfite, calcium carbonate, potassium citrate, sodium desoxycholate and disodium edatate; or at least one polymer selected from the group consisting of polyethylene glycol, polysorbate, polyoxypropylene, polyoxyethylene, polyoxyethylated fatty acid, polyvinylpyrrolidone, polyoxyl castor oil, a polyethylene-propylene glycol copolymer, polyoxyethyleneglyceroltriricinolate, polyethoxylated castor oil and polyoxyethylene sorbitan monooleate.

Preferably, the solubilizer may be at least one saccharide selected from the group consisting of mannitol, sucrose, lactose, and sorbitol; at least one alcohol selected from the group consisting of benzyl alcohol, glycerol, and ethanol; at least one acid selected from the group consisting of an organic acid such as citric acid, an amino acid such as L-arginine, and a fatty acid such as stearic acid; at least one salt selected from the group consisting of sodium bicarbonate, sodium carbonate, sodium citrate, sodium hydroxide, sodium acetate, sodium chloride, sodium borate, sodium sulfite, calcium carbonate, potassium citrate, and sodium citrate; or at least one polymer selected from the group consisting of polyoxyl castor oil, polyoxyethylene sorbitan monooleate, sorbitan trioleate or a polyethylene-propylene glycol copolymer.

More preferably, the solubilizer may be sodium bicarbonate, sodium carbonate, sodium citrate, sodium hydroxide, sodium acetate, or L-Arginine.

The weight ratio of the solubilizer to the active ingredient may be in the range of 1:0.1˜3, but is not limited thereto.

The pharmaceutical composition according to the present invention has remarkably improved solubility and stability and may thus be easily formulated. Accordingly, the pharmaceutical composition according to the present invention may be formulated into oral dosage forms such as powders, granules, tablets, capsules, suspensions, emulsions, syrups and aerosols, external applications, suppositories, solid injections or sterile injection solutions. Particularly useful is a formulation in injection solution form.

Typically, an injection dosage form may be provided as a powder form including drug powder and a solid or liquid injection form via lyophilization. Furthermore, injections, which are in powder form that is simply mixed with an excipient or are in lyophilization form, are a dosage form able to effectively supply a drug that has an active ingredient unstable in an aqueous solution while being a water-soluble material immediately dissolved before treatment. However, such a dosage form needs to undergo a dissolution procedure via pretreatment before being administered to a subject, and a check cannot be made after dissolution has occurred for the presence of microparticles which are difficult to observe with the naked eye, and also, vessel pain, vessel obstruction or phlebitis may be caused due to precipitations formed by stability problems during storage, and also, refrigeration at other than room temperature, or freezing storage conditions may be required. Moreover, the preparation conditions are complicated and the preparation time is increased due to lyophilization that occurs during the preparation process. However, because the liquid injection dosage form is provided in a liquid phase, the preparation process is relatively simple and the preparation cost may be minimized, and pretreatment before administration may be excluded. Hence, the development of a liquid injection dosage form is more preferable because of the above advantages. For the first time in the present invention, the development of a liquid injection composition comprising the pyrazino-triazine derivative, which is very stable, is possible, and in particular, this composition is stable at room temperature and is thus favorable upon storage and transport.

In the composition of the present invention, it is important to maximize the stability of the active ingredient, and the content of the active ingredient may be 95% or more, preferably, 97% or more even in long-term storage.

The composition of the present invention may further include pharmaceutically acceptable additives such as diluents, binders, disintegrants, lubricants, pH-adjusting agents, antioxidants, solubilizing agents, isotonic agents, preservatives, buffers, bulking agents, and pain-relieving agents, within the range where effects of the present invention are not impaired. In addition, the composition of the present invention can selectively use a pharmaceutically acceptable additive of various additives such as colorants, fragrances and the like, thus formulating the composition.

The range of the additives that can be used in the present invention is not limited to the above-mentioned additives, and the additive may be used in a conventional dose which can be suitably selected by those skilled in the art.

The dosage of the composition may be changed according to the weight, age, sex, health, diet, dosing time, dosing method, excretion, disease seriousness or the like of a patient. The dosage of the composition may be 0.1 to 300 mg/kg/day, preferably, 0.5 to 20 mg/kg/day.

In addition, the present invention provides a method of stabilizing a composition including at least one compound selected from the group consisting of a compound represented by Chemical Formula 1, an isomer thereof and a pharmaceutically acceptable salt thereof, the method including the step of mixing the composition with at least one stabilizer selected from the group consisting of a saccharide, an acid, a salt, an antioxidant and a polymer.

Hereinafter, the present invention will be described in more detail with reference to the following examples. These examples are set forth to illustrate the present invention, and the scope of the present invention is not limited thereto.

### Example 1

### Preparation of Composition Comprising Solubilizer

(1) Synthesis of Pyrazino-Triazine Derivative of Chemical Formula 1-1

The pyrazino-triazine derivative of Chemical Formula 1-1 was prepared using Scheme 1 above or the method disclosed in WO12/050,393, WO10/120,112, WO09/051,397 or WO09/148,192.

(2) Preparation of Compound Comprising Solubilizer

25 mg of sodium bicarbonate (NaHCO₃) as a solubilizer and 500 mg of the compound of Chemical Formula 1-1 obtained in (1) were homogeneously mixed, and then dissolved in 10 mL of water for injection, thus preparing a liquid injection dosage form.

### Examples 2 to 64

### Preparation of Compositions Comprising Solubilizer

Examples 2˜64 were carried out in the same manner as in Example 1, with the exception that the solubilizer was added in the amounts shown in Table 1 below.

TABLE 1

| | Active ingredient | Solubilizer | | | | |
|---|---|---|---|---|---|---|
| | Compound of Chemical | Sodium | Sodium | Sodium | Sodium | Sodium |
| | Formula 1-1 | bicarbonate | Carbonate) | Citrate | Hydroxide | Acetate |
| Exp. 1 | 500 | 25 | — | — | — | — |
| Exp. 2 | 500 | 50 | — | — | — | — |
| Exp. 3 | 500 | 750 | — | — | — | — |
| Exp. 4 | 500 | — | 25 | — | — | — |
| Exp. 5 | 500 | — | 35 | — | — | — |
| Exp. 6 | 500 | — | 60 | — | — | — |
| Exp. 7 | 500 | — | 750 | — | — | — |
| Exp. 8 | 500 | — | — | 25 | — | — |
| Exp. 9 | 500 | — | — | 50 | — | — |
| Exp. 10 | 500 | | | 75 | | |
| Exp. 11 | 500 | — | — | 750 | — | — |
| Exp. 12 | 500 | — | — | — | 25 | — |
| Exp. 13 | 500 | — | — | — | 50 | — |
| Exp. 14 | 500 | — | — | — | 750 | — |
| Exp. 15 | 500 | — | — | — | — | 50 |
| Exp. 16 | 500 | — | — | — | — | 60 |
| Exp. 17 | 500 | — | — | — | — | 75 |
| Exp. 18 | 500 | — | — | — | — | 750 |

| | Active ingredient | Solubilizer | | | | |
|---|---|---|---|---|---|---|
| | Compound of Chemical | | Citric | Stearic | | Benzyl |
| | Formula 1-1 | L-Arginine | acid | acid | Ethanol | alcohol |
| Exp. 19 | 500 | 100 | — | — | — | — |
| Exp. 20 | 500 | 150 | — | — | — | — |
| Exp. 21 | 500 | 200 | — | — | — | — |
| Exp. 22 | 500 | 750 | — | — | — | — |
| Exp. 23 | 500 | — | 500 | — | — | — |
| Exp. 24 | 500 | — | 750 | — | — | — |
| Exp. 25 | 500 | — | 1000 | — | — | — |
| Exp. 26 | 500 | — | — | 500 | — | — |
| Exp. 27 | 500 | — | — | 750 | — | — |
| Exp. 28 | 500 | — | — | 1000 | — | — |
| Exp. 29 | 500 | — | — | — | 500 | — |
| Exp. 30 | 500 | — | — | — | 750 | — |
| Exp. 31 | 500 | — | — | — | 1000 | — |
| Exp. 32 | 500 | — | — | — | — | 500 |
| Exp. 33 | 500 | — | — | — | — | 750 |
| Exp. 34 | 500 | — | — | — | — | 100 |

| | Active ingredient | Solubilizer | | | | |
|---|---|---|---|---|---|---|
| | Compound of Chemical | Sodim | Sodium | Sodim | Calcium | Potassium |
| | Formula 1-1 | Chloride | Borate | Sulfite | Carbonate | Citrate |
| Exp. 35 | 500 | 500 | — | — | — | — |
| Exp. 36 | 500 | 750 | — | — | — | — |
| Exp. 37 | 500 | 1000 | — | — | — | — |
| Exp. 38 | 500 | — | 500 | — | — | — |
| Exp. 39 | 500 | — | 750 | — | — | — |
| Exp. 40 | 500 | — | 1000 | — | — | — |
| Exp. 41 | 500 | — | — | 500 | — | — |
| Exp. 42 | 500 | — | — | 750 | — | — |
| Exp. 43 | 500 | — | — | 1000 | — | — |
| Exp. 44 | 500 | — | — | — | 500 | — |
| Exp. 45 | 500 | — | — | — | 750 | — |
| Exp. 46 | 500 | — | — | — | 1000 | — |
| Exp. 47 | 500 | — | — | — | — | 500 |
| Exp. 48 | 500 | — | — | — | — | 750 |
| Exp. 49 | 500 | — | — | — | — | 1000 |

| | Active ingredient | Solubilizer | | | | |
|---|---|---|---|---|---|---|
| | Compound of Chemical | Cremophor | Cremophor | | | Poloxamer |
| | Formula 1-1 | EL | ELP | Tween 80 | Span 85 | 188 |
| Exp. 50 | 500 | 250 | — | — | — | — |
| Exp. 51 | 500 | 500 | — | — | — | — |
| Exp. 52 | 500 | 750 | — | — | — | — |
| Exp. 53 | 500 | — | 250 | — | — | — |
| Exp. 54 | 500 | — | 500 | — | — | — |
| Exp. 55 | 500 | — | 750 | — | — | — |
| Exp. 56 | 500 | — | — | 250 | — | — |
| Exp. 57 | 500 | — | — | 500 | — | — |
| Exp. 58 | 500 | — | — | 750 | — | — |
| Exp. 59 | 500 | — | — | — | 250 | — |
| Exp. 60 | 500 | — | — | — | 500 | — |
| Exp. 61 | 500 | — | — | — | 750 | — |
| Exp. 62 | 500 | — | — | — | — | 250 |
| Exp. 63 | 500 | — | — | — | — | 500 |
| Exp. 64 | 500 | — | — | — | — | 750 |
| Comp. | 500 | — | — | — | — | — |
| Exp. 1 | | | | | | |

### Examples 65 to 81

### Preparation of Compositions Comprising Solubilizer and Stabilizer

A stabilizer (unit: mg) and sodium carbonate (70 mg) as a solubilizer, shown in Table 2 below, were sequentially added to the compound (500 mg) of Chemical Formula 1-1 obtained in (1) of Example 1, and they were homogeneously mixed and then dissolved in 10 mL of water for injection, thus preparing a liquid injection dosage form containing the compound of Chemical Formula 1-1.

TABLE 2

| | Active | | | | |
|---|---|---|---|---|---|
| | ingredient | | | | |
| | Compound of | Solubilizer | | | |
| | Chemical | Sod. | Stabilizer | | |
| | Formula 1-1 | Carbonate | D-Mannitol | Sucrose | D-Sorbitol |
| Exp. 65 | 500 | 70 | 1000 | — | — |
| Exp. 66 | 500 | 70 | — | 250 | — |
| Exp. 67 | 500 | 70 | — | — | 25 |

| | Active | | | | |
|---|---|---|---|---|---|
| | ingredient | | | | |
| | Compound of | Solubilizer | Stabilizer | | |
| | Chemical | Sod. | Sod. | Sod. | Sod. |
| | Formula 1-1 | Carbonate | Chloride | Citrate | Sulfite |
| Exp. 68 | 500 | 70 | 1000 | — | — |
| Exp. 69 | 500 | 70 | — | 30 | — |
| Exp. 70 | 500 | 70 | — | — | 10 |

| | Active | | | | |
|---|---|---|---|---|---|
| | ingredient | | | | |
| | Compound of | Solubilizer | Stabilizer | | |
| | Chemical | Sod. | Poloxamer | Poloxamer | Vita- |
| | Formula 1-1 | Carbonate | 188 | 407 | min C |
| Exp. 71 | 500 | 70 | 10 | — | — |
| Exp. 72 | 500 | 70 | — | 100 | — |
| Exp. 73 | 500 | 70 | — | — | 100 |

| | Active | | | | |
|---|---|---|---|---|---|
| | ingredient | | | | |
| | Compound of | Solubilizer | Stabilizer | | |
| | Chemical | Sod. | Tocoph- | Gluta- | Aspar- |
| | Formula 1-1 | Carbonat) | erol | mine | agine |
| Exp. 74 | 500 | 70 | 100 | — | — |
| Exp. 75 | 500 | 70 | — | 60 | — |
| Exp. 76 | 500 | 70 | — | — | 20 |

| | Active | | | | |
|---|---|---|---|---|---|
| | ingredient | | | | |
| | Compound of | Solubilizer | Stabilizer | | |
| | Chemical | Sod. | Sod. | D-Sor- | Sod. |
| | Formula 1-1 | Carbonate | Sulfite | bitol | Chloride |
| Exp. 77 | 500 | 70 | 10 | 25 | — |
| Exp. 78 | 500 | 70 | 10 | 50 | — |
| Exp. 79 | 500 | 70 | 10 | 100 | — |
| Exp. 80 | 500 | 70 | 10 | — | 1000 |
| Exp. 81 | 500 | 70 | 10 | 25 | 1000 |
| Comp. | 500 | 70 | | — | — |
| Exp. 2 | | | | | |

### Examples 82 to 88

### Preparation of Compositions Comprising Solubilizer and Stabilizer

A stabilizer (unit: mg) and sodium carbonate (7.42 mg) as a solubilizer, shown in Table 3 below, were sequentially added to the compound (50 mg) of Chemical Formula 1-1 obtained in (1) of Example 1, and they were homogeneously mixed and then dissolved in 1 mL of water for injection, thus preparing a liquid injection dosage form containing the compound of Chemical Formula 1-1.

TABLE 3

| | | Exp. | Exp. | Exp. | Exp. | Exp. | Exp. | Exp. |
|---|---|---|---|---|---|---|---|---|
| | — | 82 | 83 | 84 | 85 | 86 | 87 | 88 |
| Active | Compound | 50 | 50 | 50 | 50 | 50 | 50 | 50 |
| ingre- | of Chem- | | | | | | | |
| dient | ical For- | | | | | | | |
| | mula 1-1 | | | | | | | |
| Solubi- | Sod. | 7.42 | 7.42 | 7.42 | 7.42 | 7.42 | 7.42 | 7.42 |
| lizer | Carbonate | | | | | | | |
| Stabi- | D-Mannitol | 98.52 | — | — | — | 3.21 | 1.19 | 1.19 |
| lizer | Sod. | — | 89.86 | — | — | — | — | — |
| | Chloride | | | | | | | |
| | Sucrose | — | — | 96.15 | — | — | — | — |
| | Poloxamer | — | — | — | 2.19 | — | — | — |
| | 188 | | | | | | | |
| | Sod. Citrate | — | — | — | — | 2.88 | — | — |
| | Sod. Sulfite | — | — | — | — | — | 1.02 | |
| | D-Sorbitol | — | — | — | — | — | — | 10 |
| | Water for | — | 1 | 1 | 1 | 1 | 1 | 1 |
| | injection | | | | | | | |
| | (mL) | | | | | | | |

### Examples 89 to 92

### Preparation of Compositions Comprising Solubilizer and Stabilizer

A stabilizer (unit: mg) and sodium carbonate (7.42 mg) as a solubilizer, shown in Table 4 below, were sequentially added to the compound (50 mg) of Chemical Formula 1-1 obtained in (1) of Example 1, and they were homogeneously mixed and then dissolved in 1 mL of water for injection, thus preparing a liquid injection dosage form containing the compound of Chemical Formula 1-1.

TABLE 4

| | | Exp. | Exp. | Exp. | Exp. |
|---|---|---|---|---|---|
| | | 89 | 90 | 91 | 92 |
| Active ingredient | Compound of Chemical | 50 | 50 | 50 | 50 |
| | Formula 1-1 | | | | |
| Solubilizer | Sod. Carbonate | 7.42 | 7.42 | 7.42 | 7.42 |
| Stabilizer | Sod. Chloride | 44.93 | — | — | — |
| | Sucrose | — | 24.04 | — | — |
| | D-Sorbitol | | | 2.5 | — |
| | Sod. Sulfite | — | — | — | 1.02 |
| | Water for injection(mL) | 1 | 1 | 1 | 1 |

### Examples 93 to 95

### Preparation of Compositions Comprising Stabilizer

(1) Synthesis of Pyrazino-Triazine Derivative of Chemical Formula 1-3

The pyrazino-triazine derivative of Chemical Formula 1-3 was prepared using Scheme 1 above or the method disclosed in WO12/050,393, WO10/120,112, WO09/051,397 or WO09/148,192.

(2) Preparation of Compound Comprising Stabilizer

A stabilizer (unit: mg) were homogeneously mixed with 50 mg of the compound of Chemical Formula 1-3 obtained in (1), and then dissolved in 1 mL of water for injection, thus preparing a liquid injection dosage form.

TABLE 5

| | | Exp. | Exp. | Exp. |
|---|---|---|---|---|
| | | 93 | 94 | 95 |
| Active ingredient | Compound of Chemical | 50 | 50 | 50 |
| | Formula 1-1 | | | |
| Stabilizer | Sodium chloride | 45 | — | — |
| | Sucrose | — | 24 | — |
| | D-Sorbitol | — | — | 2.5 |
| | Water for injection(mL) | 1 | 1 | 1 |

### Examples 96 to 98

### Preparation of Compositions Comprising Solubilizer and Stabilizer

(1) Synthesis of Pyrazino-Triazine Derivative of Chemical Formula 1-2

The pyrazino-triazine derivative of Chemical Formula 1-2 was prepared using Scheme 1 above or the method disclosed in WO12/050,393, WO10/120,112, WO09/051,397 or WO09/148,192.

(2) Preparation of Compound Comprising Solubilizer and Stabilizer

A stabilizer (unit: mg) and sodium carbonate (0.64 mg) as a solubilizer, shown in Table 6 below, were sequentially added to the compound (4 mg) of Chemical Formula 1-2 obtained in (1), and they were homogeneously mixed and then dissolved in 1 mL of water for injection, thus preparing a liquid injection dosage form containing the compound of Chemical Formula 1-2.

TABLE 6

| | | Exp. | Exp. | Exp. |
|---|---|---|---|---|
| | | 96 | 97 | 98 |
| Active ingredient | Compound of Chemical | 4 | 4 | 4 |
| | Formula 1-2 | | | |
| Solubilizer | Sod. Carbonate | 0.64 | 0.64 | 0.64 |
| Stabilizer | Sodium chloride | 3.6 | — | — |
| | Sucrose | — | 1.92 | — |
| | D-Sorbitol | — | — | 0.2 |
| | Water for injection (mL) | 1 | 1 | 1 |

### Examples 99 to 108

### Preparation of Compositions Comprising Solubilizer and Stabilizer

(1) Synthesis of Pyrazino-Triazine Derivative of Chemical Formula 1-1

The pyrazino-triazine derivative of Chemical Formula 1-1 was prepared using Scheme 1 above or the method disclosed in WO12/050,393, WO10/120,112, WO09/051,397 or WO09/148,192.

(2) Preparation of Compound Comprising Solubilizer and Stabilizer

A stabilizer (unit: mg) and sodium carbonate (7.42 mg) as a solubilizer, shown in Table 7 below, were sequentially added to the compound (50 mg) of Chemical Formula 1-1 obtained in (1), and they were homogeneously mixed and then dissolved in 1 mL of water for injection, thus preparing a liquid injection dosage form containing the compound of Chemical Formula 1-1.

TABLE 7

| | | Exp. | Exp. | Exp. |
|---|---|---|---|---|
| | | 99 | 100 | 101 |
| Active ingredient | Compound of Chemical | 50.00 | 50.00 | 50.00 |
| | Formula 1-1 | | | |
| Solubilizer | Sod. Carbonate | 7.42 | 7.42 | 7.42 |
| Stabilizer | Sod. Chloride | 89.86 | 44.93 | 22.46 |
| | Remark | 100% | 50% | 25% |
| | | Exp. | Exp. | Exp. |
| | | 102 | 103 | 104 |
| Active ingredient | Compound of Chemical | 50.00 | 50.00 | 50.00 |
| | Formula 1-1 | | | |
| Solubilizer | Sod. Carbonate | 7.42 | 7.42 | 7.42 |
| Stabilizer | D-Mannitol | 98.52 | 49.26 | 24.63 |
| | Remark | 100% | 50% | 25% |

| | | Exp. | Exp. |
|---|---|---|---|
| | | 105 | 106 |
| Active ingredient | Compound of Chemical | 50.00 | 50.00 |
| | Formula 1-1 | | |
| Solubilizer | Sod. Carbonate | 7.42 | 7.42 |
| Stabilizer | Sod. Citrate | 3.00 | 1.50 |
| | Remark | 100% | 50% |
| | | Exp. | Exp. |
| | | 107 | 108 |
| Active ingredient | Compound of Chemical | 50.00 | 50.00 |
| | Formula 1-1 | | |
| Solubilizer | Sod. Carbonate | 7.42 | 7.42 |
| Stabilizer | Poloxamer 188 | 2.00 | 1.00 |
| | Remark | 100% | 50% |

### Examples 109 to 113

### Preparation of Compositions Comprising Solubilizer and Stabilizer

(1) Synthesis of Pyrazino-Triazine Derivative of Chemical Formula 1-1

The pyrazino-triazine derivative of Chemical Formula 1-1 was prepared using Scheme 1 above or the method disclosed in WO12/050,393, WO10/120,112, WO09/051,397 or WO09/148,192.

(2) Preparation of Compound Comprising Solubilizer and Stabilizer

A stabilizer (unit: mg) and sodium carbonate (7.42 mg) as a solubilizer, shown in Table 8 below, were sequentially added to the compound (50 mg) of Chemical Formula 1-1 obtained in (1), and they were homogeneously mixed and then dissolved in 1 mL of water for injection, thus preparing a liquid injection dosage form containing the compound of Chemical Formula 1-1.

TABLE 8

| | | Exp. | Exp. | Exp. | Exp. | Exp. |
|---|---|---|---|---|---|---|
| | | 109 | 110 | 111 | 112 | 113 |
| Active | Compound of | 50.00 | 50.00 | 50.00 | 50.00 | 50.00 |
| ingredient | Chemical | | | | | |
| | Formula 1-1 | | | | | |
| Solubilizer | Sod. Carbonate | 7.42 | 7.42 | 7.42 | 7.42 | 7.42 |
| Stabilizer | Acetyl cysteine | 10.25 | — | — | — | — |
| | Tween ® 80 | — | 100 μl | — | — | — |
| | HP-β-CD | — | | 4.05 | — | — |
| | Lactose | — | — | — | 99.53 | — |
| | monohydrate | | | | | |
| | EDTA | — | — | — | — | 7.74 |

### Comparative Example 1

### Preparation of Composition

A composition of Comparative Example 1 was prepared in the same manner as in Example 1, except that the solubilizer was not added.

### Comparative Example 2

### Preparation of Composition

A composition of Comparative Example 2 was prepared in the same manner as in Examples 65˜81, except that the stabilizer was not added.

### Experimental Example 1

### Evaluation of Solubility of the Composition of the Present Invention

(1) Methods

5 mL of water for injection was added to Examples 1˜64 and Comparative Example 1 and well mixed using a vortex and dissolved. After warming in a water bath for 20 minutes, the mixture was shaken at 37□ in the water bath for 18 hours and the supernatant was centrifuged using a centrifuge, after which the resultant supernatant was filtered using a syringe filter and analyzed using liquid chromatography.

(2) Results

The results are shown in Table 3 below.

TABLE 9

| | Solubility (mg/mL) |
|---|---|
| Exp. 1 | 17.9 |
| Exp. 2 | 50.9 |
| Exp. 3 | 66.6 |
| Exp. 4 | 54.9 |
| Exp. 5 | 57.2 |
| Exp. 6 | 55.8 |
| Exp. 7 | 65.5 |
| Exp. 8 | 13.9 |
| Exp. 9 | 48.3 |
| Exp. 10 | 58.5 |
| Exp. 11 | 66.5 |
| Exp. 12 | 46.9 |
| Exp. 13 | 55.0 |
| Exp. 14 | 65.4 |
| Exp. 15 | 51.4 |
| Exp. 16 | 56.6 |
| Exp. 17 | 57.5 |
| Exp. 18 | 65.7 |
| Exp. 19 | 32.7 |
| Exp. 20 | 56.2 |
| Exp. 21 | 57.1 |
| Exp. 22 | 65.0 |
| Exp. 23 | 31.4 |
| Exp. 24 | 34.5 |
| Exp. 25 | 36.8 |
| Exp. 26 | 29.4 |
| Exp. 27 | 30.5 |
| Exp. 28 | 31.3 |
| Exp. 29 | 21.8 |
| Exp. 30 | 23.5 |
| Exp. 31 | 25.1 |
| Exp. 32 | 34.4 |
| Exp. 33 | 36.1 |
| Exp. 34 | 37.8 |
| Exp. 35 | 40.1 |
| Exp. 36 | 41.5 |
| Exp. 37 | 40.9 |
| Exp. 38 | 25.8 |
| Exp. 39 | 29.1 |
| Exp. 40 | 30.2 |
| Exp. 41 | 20.4 |
| Exp. 42 | 19.8 |
| Exp. 43 | 21.1 |
| Exp. 44 | 25.1 |
| Exp. 45 | 26.4 |
| Exp. 46 | 28.4 |
| Exp. 47 | 22.4 |
| Exp. 48 | 21.9 |
| Exp. 49 | 21.4 |
| Exp. 50 | 35.8 |
| Exp. 51 | 37.9 |
| Exp. 52 | 39.1 |
| Exp. 53 | 36.4 |
| Exp. 54 | 39.1 |
| Exp. 55 | 40.4 |
| Exp. 56 | 31.2 |
| Exp. 57 | 33.4 |
| Exp. 58 | 35.2 |
| Exp. 59 | 12.4 |
| Exp. 60 | 22.4 |
| Exp. 61 | 24.2 |
| Exp. 62 | 18.1 |
| Exp. 63 | 19.4 |
| Exp. 64 | 18.9 |
| Comp. Exp. 1 | 1.8 |
| — | — |

As shown in Table 3 above, it can be ascertained that, when a solubilizer was added to the composition of the present invention, the solubility of the composition was remarkably increased.

### Experimental Example 2

### Evaluation of Stability of the Composition of the Present Invention and Evaluation of the Content of Active Ingredient According to Storage Conditions

(1) Methods

The compositions of Examples and Comparative Examples were shielded against light and left for a predetermined period of time under refrigeration, room temperature, high-temperautre severity (1 and 2) conditions shown in Table 10 below, and then the amount of the pyrazino-triazine derivative as an active ingredient was measured to evaluate stability. The amount thereof was analyzed using liquid chromatography.

TABLE 10

| | Storage Condition |
|---|---|
| Refrigeration | 5 ± 3□ |
| Room temperature | 25 ± 2□, 60 ± 5% |
| High-temperature severity -1 | 60□, 1 week |
| High-temperature severity -2 | 60□, 2 week |

Specifically, the amount of the active ingredient was evaluated as follows.

<Preparation of Reference Solution>

20 mg of each accurately-weighed standard active ingredient was dissolved in a diluting solvent in a 50 mL flask, and was then filtered using a membrane filter having a pore size of 0.45 μm to prepare a reference solution.

<Preparation of Test Solution>

Each of the compositons of Examples (or Comparative Examples) was dissolved in a diluting solvent (50% Acetonitrile) in a 50 mL flask to form 50 mL of a first dilute solution. 1 mL of the first dilute solution was further diluted with the diluting solvent to form a second dilute solution. The second dilute solution was filtered using a membrane filter having a pore size of 0.45 μm to prepare a test solution.

<Calculation of Active Ingredient Content>

$$Active gradient content ( \% ) = \frac{peak area of compound 1 - 1 in test solution}{peak area of compound 1 - 1 in reference solution} \times \frac{standard solution concentration}{test solution concentration} \times purity of standard active gradient ( \% )$$

(2) Results

1) Stability Under Refrigeration Conditions

The results are shown in Tables 11 to 13 below.

TABLE 11

| | Storage Condition | | | | | |
|---|---|---|---|---|---|---|
| | Refrigeration (5 ± 3□) | | | | | |
| Period (month) | 0 | 2 | 4 | 6 | 9 | 12 |
| Exp. 65 | 100.2 | 100.1 | 99.4 | 101.4 | 98.7 | 97.5 |
| Exp. 66 | 100.8 | 100.8 | 99.2 | 100.6 | 101.8 | 101.2 |
| Exp. 67 | 100.4 | 97.9 | 97.8 | 99.5 | 99.7 | 99.7 |
| Exp. 68 | 101.5 | 100.2 | 98.6 | 99.4 | 102.3 | 99.80 |
| Exp. 69 | 100.5 | 98.8 | 98.4 | 97.2 | 97.0 | 97.8 |
| Exp. 70 | 102.0 | 101.5 | 99.9 | 101.4 | 102.9 | 100.8 |
| Exp. 71 | 99.9 | 98.5 | 99.1 | 98.4 | 99.4 | 99.9 |
| Exp. 72 | 101.1 | 99.4 | 98.4 | 99.6 | 98.1 | 99.1 |
| Exp. 73 | 101.0 | 99.5 | 98.1 | 99.4 | 99.8 | 99.0 |
| Exp. 74 | 100.7 | 100.5 | 98.5 | 99.4 | 98.1 | 99.4 |
| Exp. 75 | 99.4 | 99.0 | 101.4 | 100.4 | 97.4 | 98.1 |
| Exp. 76 | 99.5 | 101.1 | 99.8 | 97.5 | 98.1 | 99.4 |
| Exp. 77 | 102.6 | 100.5 | 99.2 | 100.6 | 98.5 | 100.7 |
| Exp. 78 | 101.6 | 98.2 | 97.5 | 98.0 | 99.4 | 98.3 |
| Exp. 79 | 100.9 | 98.1 | 97.5 | 98.5 | 98.4 | 98.4 |
| Exp. 80 | 100.9 | 97.3 | 96.5 | 99.2 | 100.6 | 99.4 |
| Exp. 81 | 99.8 | 99.8 | 97.0 | 98.4 | 99.8 | 98.1 |
| Comparative | 100.5 | 90.4 | 65.4 | 50.1 | — | — |
| Example 2 | | | | | | |

Unit: %, Assay

TABLE 12

| | Storage Condition | | | | | | | |
|---|---|---|---|---|---|---|---|---|
| | Refrigeration (5 ± 3□) | | | | | | | |
| Period (month) | 0 | 2 | 4 | 6 | 9 | 12 | 18 | 24 |
| Exp. 89 | 101.50 | 100.23 | 98.60 | 99.39 | 102.34 | 99.80 | 99.25 | 101.07 |
| Exp. 90 | 100.81 | 100.78 | 99.19 | 100.60 | 101.83 | 101.16 | 99.60 | 101.51 |
| Exp. 91 | 101.98 | 101.49 | 99.91 | 101.39 | 102.90 | 100.83 | 100.58 | 101.73 |
| Exp. 92 | 100.37 | 97.85 | 97.83 | 99.47 | 99.74 | — | — | — |

TABLE 13

Storage Condition
Refrigeration (5 ± 3□)

| Period (week) | 0 | 4 |
|---|---|---|
| Exp. 96 | 99.57 | 99.53 |
| Exp. 97 | 99.57 | 99.52 |
| Exp. 98 | 99.58 | 99.54 |

Unit: %, Assay

It was ascertained that all the examples according to the present invention were stable under refrigeration conditions because the stability thereof was approximately 100%.

2) Stability Under Room Temperature Conditions

The results are shown in Table 14 below.

TABLE 14

Storage Condition
Room Temperature (25 ± 2□, 60 ± 5%)

| Period (month) | 0 | 2 | 4 | 6 |
|---|---|---|---|---|
| Exp. 65 | 100.2 | 99.8 | 99.4 | 97.2 |
| Exp. 66 | 100.8 | 100.2 | 97.0 | 97.9 |
| Exp. 67 | 100.4 | 97.7 | 94.7 | 95.6 |
| Exp. 68 | 101.5 | 100.0 | 96.8 | 97.0 |
| Exp. 69 | 100.5 | 98.4 | 97.8 | 96.1 |
| Exp. 70 | 102.0 | 99.7 | 97.5 | 98.4 |
| Exp. 71 | 99.9 | 98.5 | 99.2 | 96.3 |
| Exp. 72 | 101.1 | 99.1 | 97.4 | 96.1 |
| Exp. 73 | 101.0 | 99.7 | 98.4 | 97.2 |
| Exp. 74 | 100.7 | 99.8 | 97.5 | 96.4 |
| Exp. 75 | 99.4 | 98.1 | 96.8 | 95.7 |
| Exp. 76 | 99.5 | 99.9 | 98.1 | 97.1 |
| Exp. 77 | 102.6 | 100.7 | 96.6 | 97.4 |
| Exp. 78 | 101.6 | 97.5 | 97.8 | 97.7 |
| Exp. 79 | 100.9 | 96.3 | 97.4 | 97.3 |
| Exp. 80 | 100.9 | 97.1 | 98.1 | 98.2 |
| Exp. 81 | 99.8 | 97.7 | 98.2 | 96.3 |
| Comp. Exp. 2 | 100.5 | 89.0 | 48.1 | — |

Unit: %, Assay

It was ascertained that all the examples according to the present invention were remarkably stable even under room temperature conditions compared to the Comparative Example 2.

3) Stability Under High-Temperature Severity Condition-1 (60□, 1 Week)

The results are shown in Tables 15 and 16 below.

TABLE 15

| | Exp. | Exp. | Exp. | Exp. | Exp. | Exp. | Exp. |
|---|---|---|---|---|---|---|---|
| | 82 | 83 | 84 | 85 | 86 | 87 | 88 |
| Purity (%) | 98.91 | 98.51 | 97.42 | 96.97 | 98.06 | 97.87 | 97.45 |

Unit: %, Assay

TABLE 16

| | Exp. 109 | Exp. 110 | Exp. 111 | Exp. 112 | Exp. 113 |
|---|---|---|---|---|---|
| Purity (%) | 91.21 | 88.59 | 93.45 | 86.74 | 92.58 |

Unit: %, Assay

4) Stability Under High-Temperature Severity Condition-2 (60□, 2 Weeks)

The results are shown in Tables 15 and 16 below.

TABLE 17

Storage Condition
High-temperature severity (60□)

| Period (week) | 0 | 2 |
|---|---|---|
| Exp. 93 | 99.45% | 99.46% |
| Exp. 94 | 99.45% | 99.43% |
| Exp. 95 | 99.47% | 99.46% |

Unit: %, Assay

TABLE 18

Storage Condition
High-temperature severity (60□)

| Period (week) | 0 | 2 |
|---|---|---|
| Exp. 96 | 99.57 | 99.48% |
| Exp. 97 | 99.57 | 99.46% |
| Exp. 98 | 99.58 | 99.46% |

Unit: %, Assay

It was ascertained that all the examples according to the present invention were remarkably stable even under high temperature severity conditions.

### Experimental Example 3

### Evaluation of Stability of the Composition of the Present Invention and Evaluation of the Content of Related Substance According to Storage Conditions

(1) Methods

<Preparation of Reference Solution>

20 mg of each accurately-weighed standard active ingredient was dissolved in a diluting solvent in a 50 mL flask, and was then filtered using a membrane filter having a pore size of 0.45 μm to prepare a reference solution.

<Preparation of Test Solution>

Each of the compositions of Examples (or Comparative Examples) was dissolved in a diluting solvent (50% acetonitrile) in a 50 mL flask to form 50 mL of a first dilute solution. 1 mL of the first dilute solution was further diluted with the diluting solvent to form a second dilute solution. The second dilute solution was filtered using a membrane filter having a pore size of 0.45 μm to prepare a test solution.

The storage condition was the refrigeration condition show in Table 10, and the content of a related substance was analyzed using liquid chromatography. Generally, the stability of the compound of Chemical Formula 1 is deteriorated because a substituent X is converted into —OH. However, in Experimental Example 2, the degree of deterioration of the stability thereof is low, so that the evaluation of a related substance was carried out based on new degraded products produced during the stability test thereof, not based on the degree of coversion of the substituent X into —OH.

<Calculation of Related Substance Content>

$$Related substance content ( \% ) = \frac{peak area of produced related substance}{total peak area of test solution} \times 100$$

(2) Results

The long-term stability of each of the compositions of Examples 89 to 92 to sodium chloride, sodium sulfite, sucrose and sorbitol, each of which is a stabilizer that does not cause a problem of coloration, precipitation or the like for the results of Experimental Example 2 shown in Table 15, was evaluated.

1) Results of Example 89 (Sodium Chloride Added as a Stabilizer)

TABLE 19

| | Period (month) | | | | | | | |
|---|---|---|---|---|---|---|---|---|
| Class. | 0 | 2 | 4 | 6 | 9 | 12 | 18 | 24 |
| Compound of Chemical | 101.50 | 100.23 | 98.60 | 99.39 | 102.34 | 99.80 | 99.25 | 101.07 |
| Formula 1-1 | | | | | | | | |
| Related substance | | — | — | — | — | — | — | 0.15 |

(Unit: %)

2) Results of Example 90 (Sucrose Added as a Stabilizer)

TABLE 20

| | Period (month) | | | | | | | |
|---|---|---|---|---|---|---|---|---|
| Class. | 0 | 2 | 4 | 6 | 9 | 12 | 18 | 24 |
| Compound of Chemical | 100.81 | 100.78 | 99.19 | 100.60 | 101.83 | 101.16 | 99.60 | 101.51 |
| Formula 1-1 | | | | | | | | |
| Related substance | — | — | — | — | — | — | — | — |

(Unit: %)

3) Results of Example 91 (D-Sorbitol Added as a Stabilizer)

TABLE 21

| | Period (month) | | | | | | | |
|---|---|---|---|---|---|---|---|---|
| Class. | 0 | 2 | 4 | 6 | 9 | 12 | 18 | 24 |
| Compound of Chemical | 101.98 | 101.49 | 99.91 | 101.39 | 102.90 | 100.83 | 100.58 | 101.73 |
| Formula 1-1 | | | | | | | | |
| Related substance | — | — | — | — | — | — | | 0.16 |

(Unit: %)

4) Results of Example 92 (Sodium Sulfite Added as a Stabilizer)

TABLE 22

| | Period (month) | | | | |
|---|---|---|---|---|---|
| Class. | 0 | 2 | 4 | 6 | 9 |
| Compound of Chemical | 100.37 | 97.85 | 97.83 | 99.47 | 99.74 |
| Formula 1-1 | | | | | |
| Related substance | — | — | — | — | 0.22 |

(Unit: %)

As shown in Tables 19 to 22, it was ascertained that related substances were not produced for 6 months when sodium sulfite was used as a stabilizer, that related substances were not produced for 2 years when sodium chloride and a saccharide sulfite were used as a stabilizer, and that related substances were not produced for 1 year and 6 months when sucrose were used as a stabilizer. Consequently, it was ascertained that the compositions of Examples 89 to 92 were stable and did not produce related substances.

### Experimental Example 4

### Evaluation of Stability of the Composition of the Present Invention According to the Amount of Stabilizer

(1) Methods

The compositions of Examples 99 to 108 were left at 60□ for 2 weeks, and then the amounts of active ingredient and related substance in each of the compositions were measured.

(2) Results

The results are shown in Table 23 below.

TABLE 23

| | Exp. 99 | Exp. 100 | Exp. 101 |
|---|---|---|---|
| Purity (%) | 98.51 | 99.32 | 94.73 |
| | Exp. 102 | Exp. 103 | Exp. 104 |
| Purity (%) | 98.94 | 94.32 | 91.44 |
| | Exp. 105 | Exp. 106 | |
| Purity (%) | 97.24 | 92.20 | |
| | Exp. 107 | Exp. 108 | |
| Purity (%) | 98.18 | 90.44 | |

(Unit: mg)

Although the preferred embodiments according to the present invention have been disclosed for illustrative purposes, those skilled in the art will appreciate that various modifications, additions and substitutions are possible, without departing from the scope and spirit of the invention as disclosed in the accompanying claims.

What is claimed is:

1. A composition, comprising:
at least one compound selected from the group consisting of a compound represented by Chemical Formula 1 below, an isomer thereof and a pharmaceutically acceptable salt thereof, as an active ingredient,
wherein the composition comprises a solubilizer and a stabilizer,
wherein the solubilizer is one or more selected from the group consisting of a saccharide, an alcohol, an acid, a salt and a polymer and the stabilizer including one or more selected from the group consisting of a saccharide, an acid, a salt, an antioxidant, and a polymer: 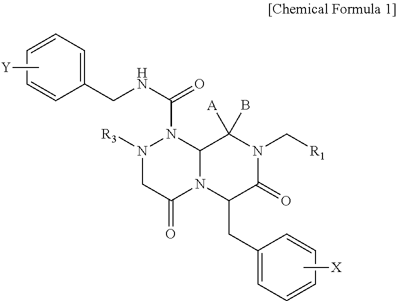
wherein R₁ is substituted or unsubstituted C3˜C10 aryl, or substituted or unsubstituted C3˜C10 heteroaryl with at least one nitrogen (N) atom;
R₃ is hydrogen, C1˜C6 alkyl, C2˜C6 alkenyl, C2˜C6 alkynyl, C3˜C10 heteroaryl with at least one nitrogen (N) atom, C3˜C10 arylalkyl, or C3˜C10 heteroarylalkyl with at least one nitrogen (N) atom;
A is hydrogen or C1˜C6 alkyl;
B is hydrogen or C1˜C6 alkyl;
X is —O—PO₃H₂, —O—SO₂NH₂, carbamate, 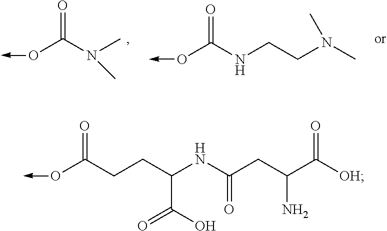
and
Y is hydrogen, C3˜C10 aryl, C3˜C10 heteroaryl with at least one nitrogen (N) atom, or C1˜C6 alkyl.

2. The composition of claim 1, wherein the substituted C3˜C10 aryl, or the substituted C3˜C10 heteroaryl with at least one nitrogen (N) atom is substituted with C1˜C6 alkyl or 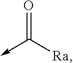
wherein Ra is hydrogen or C1˜C6 alkyl.

3. The composition of claim 1, wherein R₁ is naphthyl, quinolinyl, indolyl, indazolyl, substituted naphthyl, substituted quinolinyl, substituted indolyl or substituted indazolyl,
wherein the substituted naphthyl, the substituted the quinolinyl, the substituted indolyl and the substituted indazolyl have at least one substituent selected from C1˜C6 alkyl or 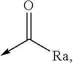
wherein Ra is hydrogen or C1˜C6alkyl;
R₃ is methyl or propenyl;
either or both of A and B is hydrogen;
X is —O—PO₃H₂; and
Y is hydrogen.

4. The composition of claim 1, wherein R₁ is 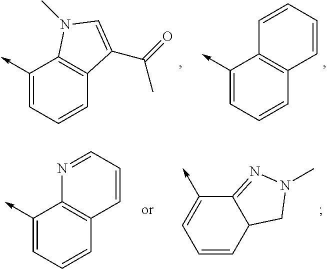
R₃ is 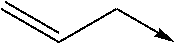
or methyl;
either or both of A and B is hydrogen;
X is —O—PO₃H₂; and
Y is hydrogen.

5. The composition of claim 1, wherein the active ingredient is a compound represented by one of Chemical Formulas 1-1 to 1-3 below:

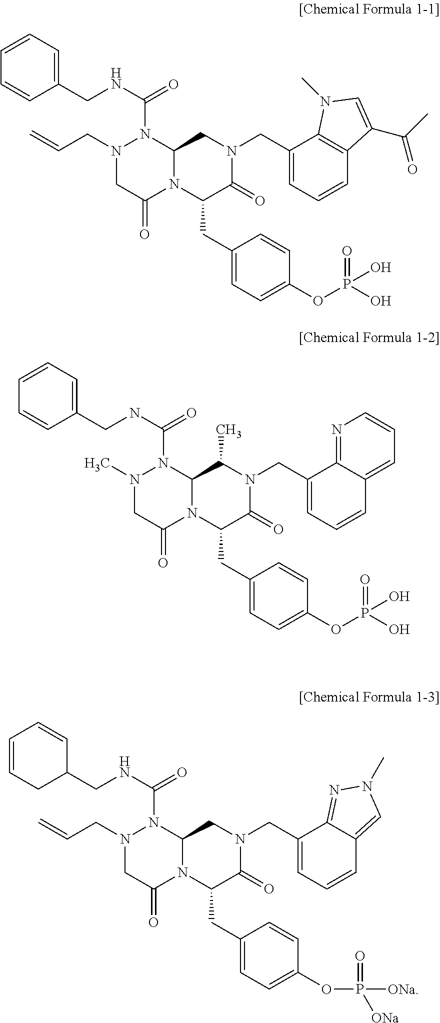

6. The composition of claim 1, wherein the active ingredient is at least one compound selected from the group consisting of a compound represented by one of Chemical Formulas 2 to 6 below, an isomer thereof and a pharmaceutically acceptable salt thereof:

| No. | Chemical Formula |
|---|---|
| 2 | 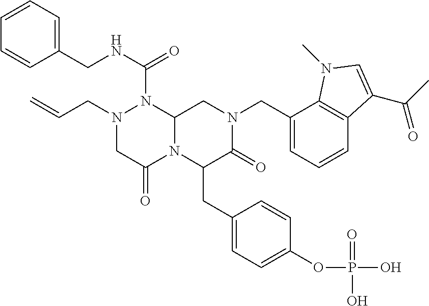 [SMILES: C=CCN1CC(=O)N2C(Cc3ccc(OP(=O)(O)O)cc3)C(=O)N(Cc3cccc4c(C(C)=O)cn(C)c34)CC2N1C(=O)NCc1ccccc1] |
| 3 | 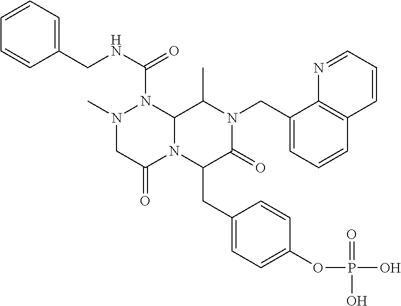 [SMILES: CC1C2N(C(=O)CN(C)N2C(=O)NCc2ccccc2)C(Cc2ccc(OP(=O)(O)O)cc2)C(=O)N1Cc1cccc2cccnc12] |
| 4 | 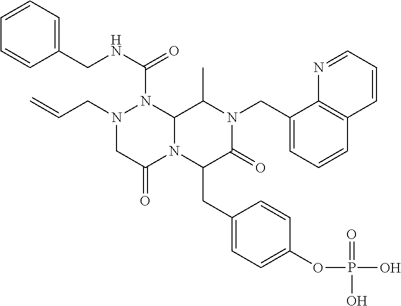 [SMILES: C=CCN1CC(=O)N2C(Cc3ccc(OP(=O)(O)O)cc3)C(=O)N(Cc3cccc4cccnc34)C(C)C2N1C(=O)NCc1ccccc1] |
| 5 | 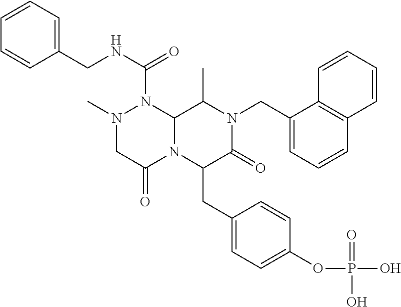 [SMILES: CC1C2N(C(=O)CN(C)N2C(=O)NCc2ccccc2)C(Cc2ccc(OP(=O)(O)O)cc2)C(=O)N1Cc1cccc2ccccc12] |
| 6 | 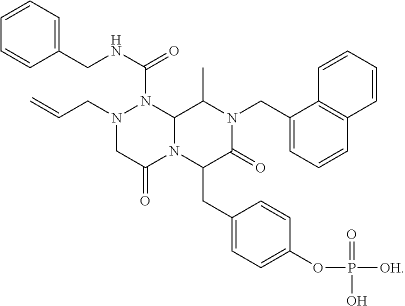 [SMILES: C=CCN1CC(=O)N2C(Cc3ccc(OP(=O)(O)O)cc3)C(=O)N(Cc3cccc4ccccc34)C(C)C2N1C(=O)NCc1ccccc1] |

7. The composition of claim 1, wherein the stabilizer comprises at least one selected from:
saccharide of mannitol, sucrose, lactose, glucose, hydroxypropyl beta cyclodextrin (HP-B-CD) or sorbitol;
acid of one or more organic acids selected from benzene sulfonic acid, benzoic acid, citric acid, lactic acid, maleic acid, methane sulfonic acid, succinic acid, tartaric acid and gentisic acid, one or more inorganic acids selected from hydrochloric acid, phosphoric acid, hydrogen bromide and sulfuric acid, one or more amino acids selected from glutamine, asparagine, leucine, glycine, isoleucine, threonine, phenylalanine, histidine, cysteine and lycine, or fatty acids;
salt of sodium chloride, sodium citrate, sodium sulfite, calcium chloride, disodium edentate, dibasic sodium phosphate, dibasic sodium phosphate dihydrate, monobasic sodium phosphate dihydrate, sodium bicarbonate, disodium succinate, gentisic acid ethanolamine, ammonium hydroxide, sodium benzoate, sodium dithionite, sodium glutamate, sodium lactate, sodium metabisulfite, sodium tartrate, sodium thioglycolate or zinc chloride;
antioxidant of acetone sodium bisulfate, sodium bisulfate, butylated hydroxyl anisole (BHA), butylated hydroxyl toluene (BHT), sodium formaldehyde sulfoxylate, monothioglycerol (thioglycerol), propyl gallate, vitamin C, ethylenediamine tetraacetic acid (EDTA) or tocopherol; and
polymer of polyethylene glycol, polysorbate, polyoxypropylene, a polyethylene-propylene glycol copolymer or polyoxyethylene sorbitan monooleate.

8. The composition of claim 1, wherein the solubilizer comprises at least one selected from:
saccharide of mannitol, sucrose, lactose, sorbitol, microcrystalline cellulose, or hydroxypropyl beta-cyclodextrin (HP-B-CD);
alcohol of benzyl alcohol, glycerol, isopropanol, propylene glycol, or ethanol;
acid of one or more organic acids selected from citric acid and lactic acid, hydroxychloric acid, one or more amino acids selected from L-arginine and L-glycine, or stearic acid;
salt of sodium bicarbonate, sodium carbonate, sodium citrate, sodium hydroxide, sodium acetate, sodium chloride, sodium borate, sodium sulfite, calcium carbonate, potassium citrate, sodium desoxycholate, or disodium edatate; and
polymer of polyethylene glycol, polysorbate, polyoxypropylene, polyoxyethylene, polyoxyethylated fatty acid, polyvinylpyrrolidone, polyoxyl castor oil, a polyethylene-propylene glycol copolymer, polyoxyethyleneglyceroltriricinolate, polyethoxylated castor oil, or polyoxyethylene sorbitan monooleate.

9. The composition of claim 1, wherein the stabilizer is at least one selected from the group consisting of a saccharide, a sodium salt and a polyethylene-propylene glycol copolymer.

10. The composition of claim 9, wherein the saccharide is at least one selected from the group consisting of mannitol, sucrose and sorbitol, and the sodium salt is at least one selected from the group consisting of sodium chloride, sodium citrate and sodium sulfite.

11. The composition of claim 1, wherein the stabilizer is at least one selected from the group consisting of sodium chloride, sucrose, mannitol and sorbitol.

12. The composition of claim 1, wherein the stabilizer is sucrose.

13. The composition of claim 1, wherein a weight ratio of the active ingredient to the stabilizer is 1:0.02˜30.

14. The composition of claim 1, wherein the composition is a liquid injection dosage form.

15. A method of stabilizing a composition comprising at least one compound selected from the group consisting of a compound represented by Chemical Formula 1 below, an isomer thereof and a pharmaceutically acceptable salt thereof, as an active ingredient,
comprising the step of mixing the composition with at least one stabilizer selected from the group consisting of a saccharide, an acid, a salt, an antioxidant and a polymer: 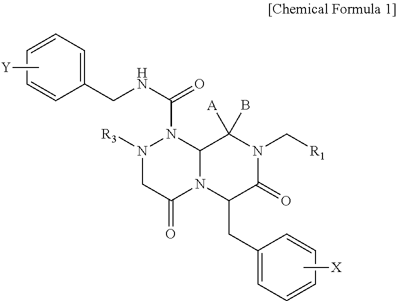
wherein R₁ is substituted or unsubstituted C3˜C10 aryl, or substituted or unsubstituted C3˜C10 heteroaryl with at least one nitrogen (N) atom;
R₃ is hydrogen, C1˜C6 alkyl, C2˜C6 alkenyl, C2˜C6 alkynyl, C3˜C10 heteroaryl with at least one nitrogen (N) atom, C3˜C10 arylalkyl, or C3˜C10 heteroarylalkyl with at least one nitrogen (N) atom;
A is hydrogen or C1˜C6 alkyl;
B is hydrogen or C1˜C6 alkyl;
X is —O—PO₃H₂, —O—SO₂NH₂, carbamate, 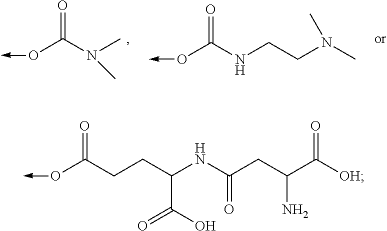
and
Y is hydrogen, C3˜C10 aryl, C3˜C10 heteroaryl with at least one nitrogen (N) atom, or C1˜C6 alkyl.
